(19) United States
(12) Patent Application Publication
(10) Pub. No.: US 2021/0275702 A1
(43) Pub. Date: Sep. 9, 2021

(54) Disinfecting Light Fixture

(71) Applicant: Violume Inc., Round Rock, TX (US)

(72) Inventor: David W. Parkansky, Round Rock, TX (US)

(73) Assignee: Violume Inc., Round Rock, TX (US)

(21) Appl. No.: 17/189,541

(22) Filed: Mar. 2, 2021

Related U.S. Application Data
(60) Provisional application No. 62/985,516, filed on Mar. 5, 2020.

(51) Int. Cl. A61L 2/10; A61L 2/24; A61L 2/26; F21V 7/00; F21V 1/08

(52) U.S. Cl. CPC A61L 2/10; A61L 2/24; A61L 2/26; A61L 2202/25; F21V 1/08; A61L 2202/11; A61L 2202/14; F21V 7/0025

(57) ABSTRACT

A disinfecting light fixture and method of disinfecting a room are provided. The disinfecting light fixture includes a housing that houses one or more light-producing element sand one or more UVC irradiation elements in separate compartments. The housing includes panels associated with the light-producing element and the one or more UVC irradiation elements that can be opened and closed to expose the light-producing element or the one or more UVC irradiation elements at selected times, and sensors that can detect heat or motion indicating the presence of a human or animal in the room and can activate the opening and closing of panels in response to such detection.

### RELATED APPLICATIONS

The present application claims the benefit of U.S. Provisional Application Ser. No. 62/985,516, filed on Mar. 5, 2020, the disclosure of which is incorporated herein by reference.

### FIELD OF THE INVENTION

The present application relates to light fixtures, and more specifically to medical grade light fixtures that include an integrated ultraviolet irradiation module that can be selectively used to disinfect room surfaces and airborne pathogens. This disclosure allows for one device to provide light to a particular room or space while also providing the ability to disinfect a room or space easily via UVC.

### BACKGROUND

Light fixtures are a standard part of medical offices and other related facilities. Additionally, ultraviolet irradiation may disinfect surfaces within a space without physically contacting each space. Ultraviolet irradiation may also disinfect the air within a space. Thus, ultraviolet irradiation is a good choice for disinfecting a room or area quickly and efficiently.

However, disinfecting surfaces or the air using ultraviolet irradiation should not be performed when human beings or other animals are in the area. Additionally, ultraviolet irradiation requires a separate fixture from a standard lighting fixture typically used in medical offices. Thus, a fixture is needed with the capability to both light a room or area while occupied and disinfect a room or area with ultraviolet irradiation.

### SUMMARY

The present disclosure may include a disinfecting light fixture comprised of one or more light-producing elements and one or more ultraviolet (UVC) irradiation elements. UVC radiation is the highest energy portion of the ultraviolet radiation spectrum, having a wavelength of about 190 to about 290 nm, and is useful for reducing the spread of bacteria and viruses. However, UVC radiation must be artificially produced and is most effective when the bacteria or virus is directly exposed to the radiation. The light-producing element of the disinfecting light fixture may therefore be used while a room or area is occupied by human beings or animals, while the UVC irradiation element may be used when the room or area is empty of human beings and animals. The UVC irradiation element may be disposed behind panels in the housing of the disinfecting light fixture such that the panels are opened when the UVC irradiation element is in operation. The disinfecting light fixture may be controlled by a microprocessor connected to any suitable user device, including a computer, smart phone, tablet, or wall panel. In addition to operating the disinfecting light fixture, the microprocessor may include software that collects data from sensors positioned within the light fixture and around the room. The software may not allow the UVC irradiation element to operate when the sensors detect a human being or other animal within the room or area to be disinfected. Additionally, the software may use the data it collects to report on the diagnostics of the disinfecting light fixture and provide information to a user regarding use and remaining life span of the fixture.

The disinfecting light fixture may include a housing with a portion configured to include the light-producing element, a portion configured to include the UVC irradiation element, and panels. The light-producing element and UVC irradiation element may be mounted within the housing. The panels may be configured so that the panels and housing fully enclose the UVC irradiation element when the panels are closed. The light fixture may also include reflectors that evenly disperse ultraviolet radiation produced by the UVC irradiation element throughout a space, a microcontroller that may control the opening and closing of the panels, sensors that may detect movement so that the UVC irradiation element does not operate when movement is detected, and motors that may open and close the panels.

A method of disinfecting a room may include providing a disinfecting light fixture that has light-producing element and an UVC irradiation element, providing a microcontroller that is capable of controlling the light-producing element and the UVC irradiation element, and providing a sensor that can detect the presence of a human being within the room. The microcontroller may turn the UVC irradiation element off when the sensor detects the presence of a human being within the room. The method may also include providing a panel that covers the UVC irradiation element and using the panel to cover the UVC irradiation element when the sensor detects the presence of a human being within the room.

In one embodiment, the invention is therefore directed to a disinfecting light fixture that includes:
a housing, the housing including a portion configured to include a light-producing element, a portion configured to include a UVC irradiation element, and panels;
one or more light-producing elements mounted within the housing; and
one or more UVC irradiation elements mounted within the housing;
wherein the panels are configured such that the panels and housing enclose the one or more UVC irradiation elements when the panels are closed.

In another embodiment, the invention is directed to a method of disinfecting a room that includes the following steps:
providing a disinfecting light fixture having one or more light-producing elements and one or more UVC irradiation elements;
providing a microcontroller that is capable of controlling the one or more light-producing elements and the one or more UVC irradiation elements; and
providing a sensor that can detect the presence of a human being within the room;
wherein the microcontroller deactivates the one or more UVC irradiation elements when the sensor detects the presence of a human being within the room.

In another embodiment, the invention is directed to a disinfecting light fixture for use in a room that includes:
a housing, the housing including a portion configured to include one or more light-producing elements, a portion configured to include one or more UVC irradiation elements, and panels;
one or more light-producing elements mounted within the housing;
one or more UVC irradiation elements mounted within the housing; and
sensors that detect motion and/or heat emanating from a human being or animal in the room;
wherein the sensors activate a closing of at least one panel to enclose the one or more UVC irradiation elements when motion and/or heat are detected.

### BRIEF DESCRIPTION OF THE DRAWINGS

FIG. 1 is a perspective view of a disinfecting light fixture with open panels.

FIG. 2 is a perspective view of a disinfecting light fixture with closed panels.

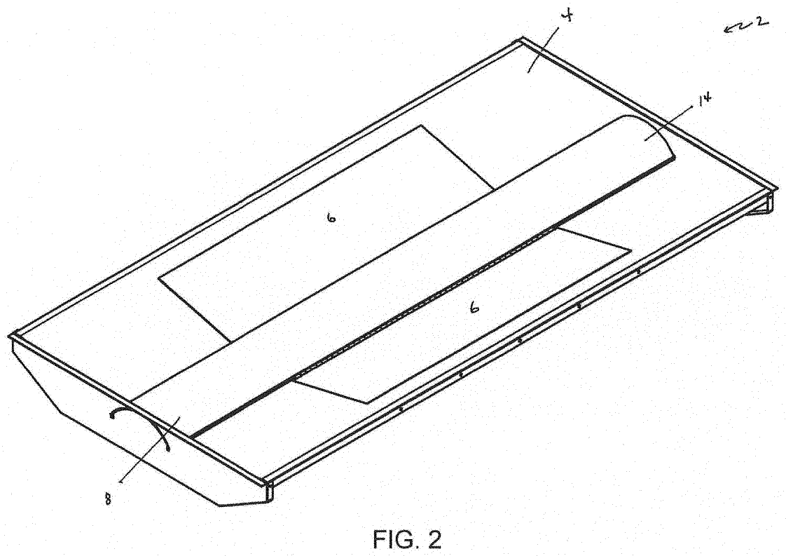

FIG. 3 is a perspective view of the back side of a disinfecting light fixture.

FIG. 4 is a bottom view of a disinfecting light fixture.

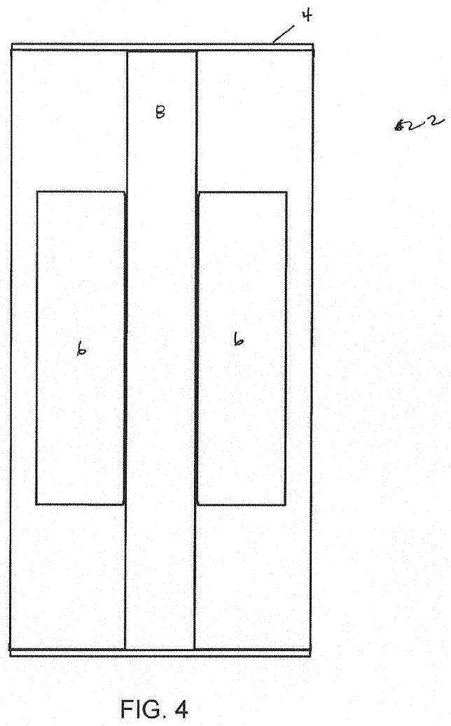

FIG. 5 is a side view of a disinfecting light fixture.

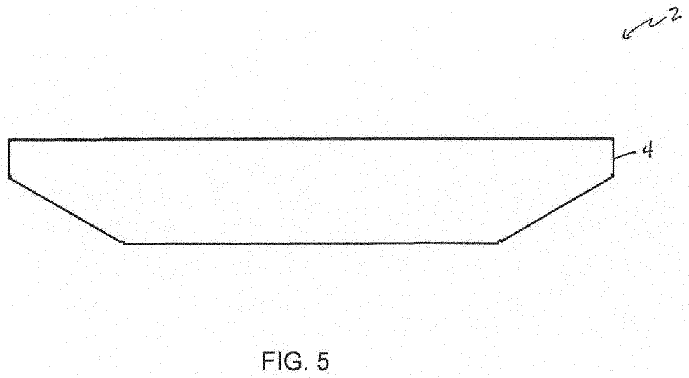

FIG. 6 is a bottom view of a disinfecting light fixture including sensors and a user device.

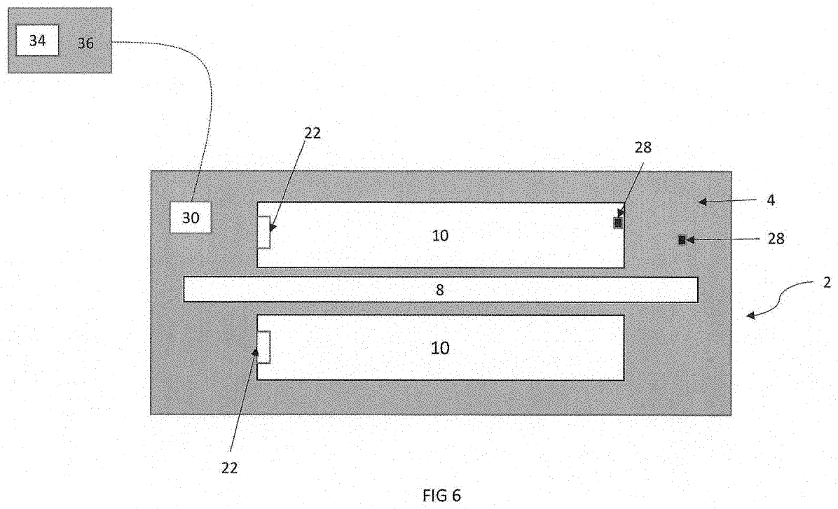

FIG. 7 is a side view of a light fixture shown mounted on the ceiling of a room.

### DETAILED DESCRIPTION

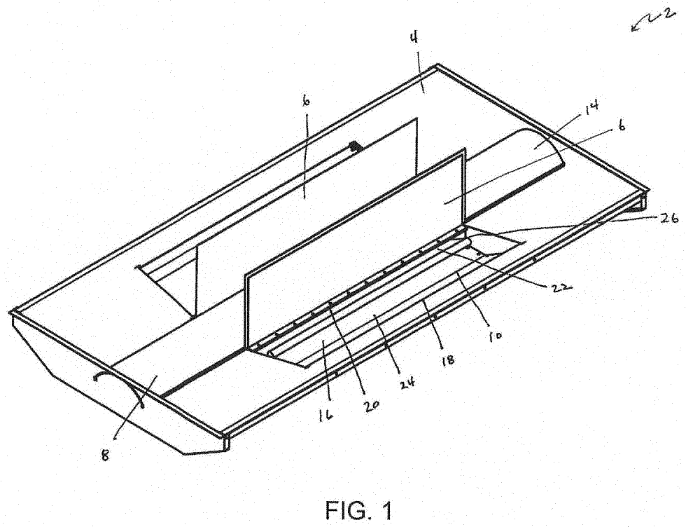

As shown in FIG. 1, a disinfecting light fixture 2 may include a housing 4, panel 6, one or more light producing elements 8, part of which may be disposed on the external surface of a panel 6, and one or more UVC irradiation elements 10. Disinfecting light fixture 2 may be mounted on any suitable surface, including a ceiling, wall, floor, post, or any other structural component that is capable of receiving a light fixture. The disinfecting light fixture 2 may also be mounted in any suitable way, including recessed, flush, or surface mounted. In one embodiment, the disinfecting light fixture can be mounted to a ceiling or another suitable surface in a room and can include an adjustment mechanism for varying the distance between the disinfecting light fixture and the ceiling or other surface. This way, when the one or more UVC irradiation elements are activated, they may be positioned closer to the surface(s) that are to be disinfected.

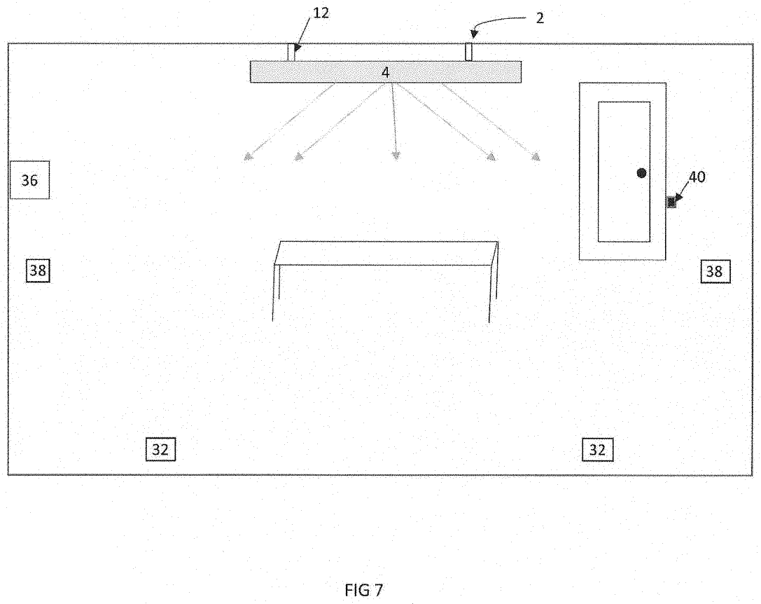

Housing 4 may be formed of any suitable material, including aluminum, steel, wood, or polymer. Housing 4 may be powder coated or painted to medical grade standards prior to assembly. The housing 4 may be configured to house the light producing element(s) 8, UVC irradiation element(s) 10, and panel 6. Housing 4 may also include a mounting portion 12 which interfaces with the ceiling, wall, post, or other structural component to which the disinfecting light fixture 2 may be mounted. As shown in FIG. 7, in one embodiment, mounting portion 12 is configured to allow a user to move disinfecting light fixture 2 closer to and further away from the surface upon which disinfecting light fixture 2 is mounted. The panel 6 may be configured to cover the one or more UVC irradiation elements 10 when they are not in use. Light producing element(s) 8 may be positioned within housing 4 and extend over the external surface of panel 6 as well as portions of the housing not covered by panel 6. The housing 4 may include one or more of the panels 6, the one or more UVC irradiation elements 10, and the one or more light producing elements 8.

The light producing element(s) 8 may be formed of light emitting diodes (LED), incandescent light bulbs, or any other device that is capable of producing white or visible light. The light producing element(s) 8 are designed to act as at least a portion of the primary light source within a room or area. The light producing element(s) 8 may be turned off and on by any mode, including but not limited to hard-wired, wirelessly, and as triggered by sensors. The light producing element(s) 8 may also be dimmed to any level from 100% output to 0% output. The light producing element(s) may also be able to be tuned to change the color temperature of the light. In one example, the color temperature of the light may be tuned within the range of 2,000 K to 7,000 K. The light producing element(s) may be mounted to an aluminum channel 14 within the housing 4 which may act as both an electrical chamber cover and a heat sink.

The one or more UVC irradiation elements 10 can be formed as thin, longitudinal bulbs as shown or can be round, circular, spiral of have any suitable configuration. The UVC irradiation element(s) 10 can be designed to produce and emit UVC light within the UVC wavelength spectrum of about 190 nm to about 290 nm, suitably about 240 nm to about 270 nm, for example, about 254 nm. The UVC radiation emitted from the UVC irradiation element(s) 10 should have sufficient intensity to produce an instantaneous UVC intensity of at least about 0.29 mW/cm² at a distance of six feet, and at least about 0.15 mW/cm² at a distance of six feet, six inches. Cumulative UVC exposure (expressed in mJ/cm²) is calculated by multiplying the instantaneous UVC intensity (expressed in mW/cm²) by the number of seconds. After an exposure time of five minutes, the UVC intensity of at least about 0.29 mW/cm² will result in a cumulative UVC exposure of at least about 87 mJ/cm² at a distance of six feet and at least about 45 mJ/cm² at a distance of six feet, six inches. The UVC intensity varies inversely with the square of the distance from the UVC irradiating element(s), according to the following equation:

I₂/I₁=d₂²/d₁²
where d₂ and d₁ are two distances from the UVC source,
I₂ is the UVC intensity at distance d₂, and
I₁ is the UVC intensity at distance d₁.
   The foregoing equation explains why it is advantageous to be able to place the UVC irradiation element(s) reasonably close to the surface(s) being disinfected and to be able to move the disinfecting light fixture at varying distances from the ceiling or other mounting surface. The UVC intensity drops quickly as the distance between the surface(s) being disinfected and the UVC irradiating element is increased. The one or more UVC irradiation elements 10 can be any suitable element(s) that produce UVC irradiation of the desired intensity, including without limitation low pressure mercury bulbs, low pressure krypton bulbs, and UVC light emitting diodes.

The UVC intensity emitted from the UVC irradiation element(s) should be sufficient to inactivate common pathogens and viruses that may be present on the operating tables, examination tables, chairs, cabinets, and other surface(s) being disinfected. One way of determining the effectiveness of a given UVC intensity at a given distance if to measure its effectiveness in inactivating Clostridium difficile spores present on a target surface at a given distance. Clostridium difficile is a Gram-positive anaerobic bacterium that forms endospores when exposed to hostile conditions. The endospores are highly resistant to chemical and environmental conditions. Clostridium difficile is extremely common and is found almost anywhere, including in soil. Clostridium difficile shows optimal growth in human blood in the absence of oxygen at human body temperature and is responsible for a variety of common ailments and infections. Because of their resistance to hostile conditions, the Clostridium difficile spores are excellent subjects for the evaluation of UVC irradiation applied at varying distances and times to inactivate the spores. Put another way, UVC intensities and exposures that are sufficient to inactivate the Clostridium difficile spores will also be sufficient (or more than sufficient) to inactivate virtually all common pathogens that might inhabit the target surfaces. The one or more UVC irradiation elements should emit UVC at a sufficient intensity so that a sample of Clostridium difficile spores placed on a target surface six feet away will be at least about 90% inactivated after 5 minutes of exposure and at least about 99% inactivated after 10 minutes of exposure. Using UVC irradiation elements that emit UVC at a wavelength of 254 nm, an instantaneous UVC radiation intensity of 0.29 mW/cm² at a distance of six feet, a sample containing cultured Clostridium difficile endospores was 94.9% inactivated after 5 minutes of exposure (reflecting a total UVC exposure of 87 mJ/cm²) and 99.52% inactivated after 10 minutes of exposure (reflecting a total UVC exposure of 174 mJ/cm²). When the experiment was repeated using a distance of 6 feet, 6 inches and an instantaneous UVC radiation intensity of 0.15 mW/cm², the sample containing cultured Clostridium difficile endospores was 48.44% inactivated after 5 minutes of exposure (reflecting a total UVC exposure of 45 mJ/cm²) and 89.97% inactivated after 10 minutes of exposure (reflecting a total UVC exposure of 90 mJ/cm²).

While the foregoing conditions are sufficient to inactivate a substantial quantity of Clostridium difficile endospores, and can be adjusted to inactivate substantially all of these endospores (e.g., at 6-foot spacing and 10 minutes of exposure), even the milder of these conditions is more than sufficient to inactivate other types of bacteria. For example, the same experiments were performed using cultured samples of Staphylococcus aureus bacteria. Using an instantaneous UVC radiation intensity of 0.29 mW/cm² at a distance of six feet, a sample containing cultured Staphylococcus aureus bacteria was greater than 99.9994% inactivated after 5 minutes of exposure (reflecting a total UVC exposure of 87 mJ/cm²) and greater than 99.9994% inactivated after 10 minutes of exposure (reflecting a total UVC exposure of 174 mJ/cm²). When the experiment was repeated using a distance of 6 feet, 6 inches and an instantaneous UVC radiation intensity of 0.15 mW/cm², the sample containing Staphylococcus aureus bacteria was 99.96% inactivated after 5 minutes of exposure (reflecting a total UVC exposure of 45 mJ/cm²) and 99.98% inactivated after 10 minutes of exposure (reflecting a total UVC exposure of 90 mJ/cm²). Other types of bacteria that can be substantially or entirely inactivated using the disinfecting light fixture include without limitation Bacillus subtilis spores, Aeromonas hydrophila, Aeromonas salmonicida, Campylobacter jejuni, Citrobacter diversus, Citrobacter freundii, Escherichia coli, Halobacterium elongata, Halobacterium salinarum, Klebsiella pneumoniae, Klebsiella terrigena, Legionella pneumophila, Pseudomonas stutzeri, various types of Salmonella, Shigella dysenteria, Shigella sonnei, Staphylococcus aureus, Streptococcus faecalis, Vibrio anguillarum, Vibrio cholerae, Vibrio natriegens, Yersinia enterocolitica and Yersinia ruckeri. Other types of microorganisms, including various protozoa and viruses, can be deactivated using the disinfecting light fixture of the invention.

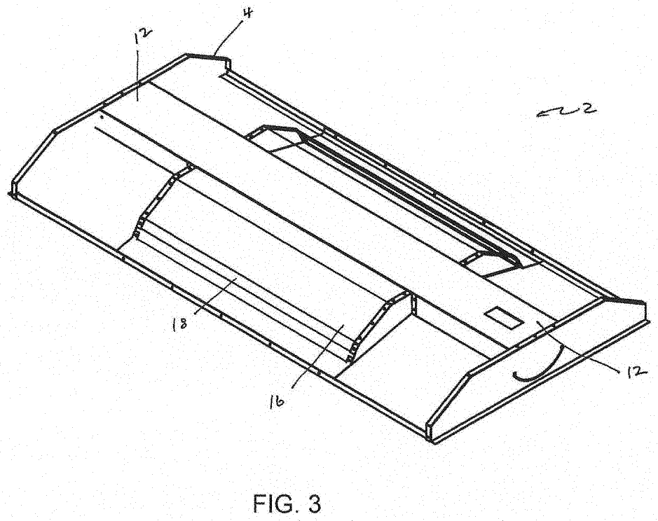

Referring to FIGS. 1 and 3, a germicidal module 16 may include a UVC lamp 26 which, in turn, includes one or more UVC irradiation elements 10 and a panel 6. The germicidal module 16 may further include a metallic enclosure 18, door opening mechanisms 20, motors 22, UV optical reflectors 24, and internal sensors 28. The germicidal module 16 may be securely fastened to the housing 4. Door opening mechanisms 20 and motors 22 may work together to open and close panels 6 as necessary to expose and recover the one or more UVC irradiation elements 10. Door opening mechanisms 20 and motors 22 may be controlled by a micro controller circuit board 30. The micro controller circuit board 30 may be mounted within housing 4. The panels 6 may be controlled by the micro controller circuit board 30 and may oscillate to reflect and increase UVC radiation to surrounding walls and surfaces. In one example, the panels 6 may oscillate from 0 to 30 degrees.

UV optical reflectors 24 may be positioned and shaped to direct the UVC radiation produced by the one or more UVC irradiation elements 10 away from the germicidal module 16 to reach as much of the room or area as possible. The UV optical reflectors 24 may also increase the efficacy of the UVC output. Internal sensors 28 may be positioned to provide information to a data processing unit regarding the position of panels 6. Internal sensors 28 may also detect motion, heat, sound, and any other indicator that human beings or other animals are in the room. As discussed more fully below, when internal sensors 28 detect motion, heat, sound, or any other indicator that a human being or animal is in the room, germicidal module will be prevented from operating. An internal sensor 28 may also measure the intensity of the UV light emitted from UVC lamp 26 in order to determine whether the UVC lamp requires maintenance.

The disinfecting light fixture may also include UVC radiation detectors 32. These UVC radiation detectors 32 may be placed within the room or area with disinfecting light fixture 2 such that the UVC radiation detectors 32 may measure the amount and intensity of UVC radiation striking the detector's surface within the space.

The disinfecting light fixture 2 may have at least two modes of operation. In the first mode of operation, the panels 6 of the light fixture 2 are closed, fully covering the one or more UVC irradiation elements 10 located behind the panels 6. In this mode, the disinfecting light fixture 2 may be used as an ordinary light fixture, illuminating the room or area where disinfecting light fixture 2 is located. In the second mode of operation, the panels 6 may be opened to uncover the one or more UVC irradiation elements 10. The one or more UVC irradiation elements 10 may then be used to disinfect a room or area.

The disinfecting light fixture 2 may be controlled by a computer circuit board 34 and microcontroller 30. The microcontroller 30 may accept input from all of the internal sensors 28, radiation detectors 32, and other electrical components of the disinfecting light fixture 2. The computer circuit board 34, microcontroller 30, and disinfecting light feature 2 may all be operated through a local network using Bluetooth, ZigBee, wireless internet, or any other secure form of communication. The microcontroller board 30 may control all operational functions of the disinfecting light fixture 2, and may be controlled from any user device 36, including but not limited to a desktop computer, laptop computer, smart phone, handheld touch pad, and/or wall mounted touch pad that may be paired to the disinfecting light fixture 2. The disinfecting light fixture 2 may be controlled by more than one user device 36 as desired.

The software controlling the disinfecting light fixture 2 may turn the light producing element 8 on and off, open and close panels 6, and turn the one or more UVC irradiation elements 10 on and off. Preferably, the one or more UVC irradiation elements 10 may be turned on when panels 6 are open, and light producing element 8 may not be turned on when panels 6 are open. The software may allow for the one or more UVC irradiation elements 10 to be turned on for a set period of time, after which the one or more UVC irradiation elements 10 turn off and the panels 6 close. External sensors 38 may monitor the room or area where disinfecting light fixture is placed, and, when the presence of a human being or animal is detected within the room or area, may prevent the one or more UVC irradiation elements 10 from turning on. The present disclosure may also include door sensors 40. In some embodiments, when doors sensors 40 detect that a door is open, the software may prevent a user from turning on the one or more UVC irradiation elements 10. Additionally, in some embodiments, when door sensors 30 detect that a door is opened during operation of the one or more UVC irradiation elements 10, the software may automatically turn off the UVC irradiation element(s) 10.

The software may also collect data on the operation of the disinfecting light fixture 2, including but not limited to the time of operation of the one or more UVC irradiation elements 10, number of times the panels 6 are opened and closed, total usage and remaining life of the one or more UVC irradiation elements 10, total usage and remaining life of the light producing elements 8, the number of motor 22 operating cycles and life expectancy, and any other data points defined by a user. The software may provide the ability for the disinfecting light fixture 2 to include a self-diagnostic functionality. The data may be collected from light producing elements 8, internal sensors 28, external sensors 38, radiation detectors 32, UVC irradiation elements 10, panels 6, user device 36 controls, and the operating profile of the wireless network. This data may be sent to a reporting system that may track and evaluate each component based on operating cycles, life expectancy, and any other feature or function deemed critical to the end user.

The embodiments of the invention described herein are exemplary. Various modifications and improvements can be made without departing from the spirit and scope of the invention. The scope of the invention is indicated by the appended claims, and all changes that fall within the meaning and range of equivalents are intended to be embraced therein.

1. A disinfecting light fixture for use in a room, comprising:
a housing, the housing including a portion configured to include one or more light-producing elements, a portion configured to include one or more UVC irradiation elements, and panels;
one or more light-producing elements mounted within the housing;
sensors that detect motion and/or heat from a human being or animal in the room; and
one or more UVC irradiation elements mounted within the housing;
wherein the panels are configured such that the panels and housing enclose the one or more UVC irradiation elements when the panels are closed, and the sensors activate a closing of at least one panel when motion and/or heat are detected.

2. The disinfecting light fixture of claim 1, further comprising reflectors that are configured to evenly disperse ultraviolet radiation produced by the one or more UVC irradiation elements throughout a space.

3. The disinfecting light fixture of claim 1, wherein the one or more UVC irradiation elements produce sufficient UVC radiation to inactivate at least about 90% of Clostridium difficile endospores in 5 minutes at a distance of six feet.

4. The disinfecting light fixture of claim 1, wherein the one or more UVC irradiation elements produce sufficient UVC radiation to inactivate at least about 99% of Clostridium difficile endospores in 10 minutes at a distance of six feet.

5. The disinfecting light fixture of claim 1, wherein the one or more UVC irradiation elements produce sufficient UVC radiation to provide an instantaneous UVC reading of at least about 0.29 mW/cm² at a distance of six feet.

6. The disinfecting light fixture of claim 1, wherein the one or more UVC irradiation elements produce sufficient UVC radiation to provide an instantaneous UVC reading of at least about 0.15 mW/cm² at a distance of six feet, six inches.

7. The disinfecting light fixture of claim 1, further comprising a microcontroller, the microcontroller configured to control an opening and closing of the panels.

8. The disinfecting light figure of claim 1, wherein when the sensors detect movement the one or more UVC irradiation elements are blocked and/or deactivated.

9. The light fixture of claim 1, further comprising motors, wherein said motors cause the panels to open and close.

10. A method of disinfecting a room, comprising the steps of:
providing a disinfecting light fixture including one or more UVC irradiation elements;
providing a microcontroller that is capable of controlling the one or more UVC irradiation elements;
providing a sensor that can detect the presence of a human being within the room;
wherein the microcontroller deactivates the one or more UVC irradiation elements when the sensor detects the presence of a human being within the room.

11. The method of claim 10, further comprising the step of:
providing a panel capable of covering the one or more UVC irradiation elements; and
using the panel to block the one or more UVC irradiation elements when the sensor detects the presence of a human being within the room.

12. The method of claim 10, further comprising the step of emitting sufficient ultraviolet radiation from the one or more UVC irradiation elements to produce an instantaneous UVC intensity of at least about 0.29 mW/cm² at a distance of six feet.

13. The method of claim 10, further comprising the step of emitting sufficient ultraviolet radiation from the one or more UVC irradiation elements to produce an instantaneous UVC intensity of at least about 0.15 mW/cm² at a distance of six feet, six inches.

14. The method of claim 10, further comprising the step of emitting sufficient ultraviolet radiation from the one or more UVC irradiation elements to produce a cumulative UVC dose of at least about 87 mJ/cm² at a distance of six feet and an exposure time of five minutes.

15. The method of claim 10, further comprising the step of emitting sufficient ultraviolet radiation from the one or more UVC irradiation elements to produce an instantaneous UVC intensity of at least about 45 mJ/cm² at a distance of six feet, six inches and an exposure time of ten minutes.

16. A disinfecting light fixture for use in a room, comprising:
a housing, the housing including a portion configured to include one or more light-producing elements, a portion configured to include one or more UVC irradiation elements, and panels;
one or more light-producing elements mounted within the housing;
one or more UVC irradiation elements mounted within the housing; and
sensors that detect motion and/or heat from a human being or animal in the room;
wherein the sensors activate a closing of at least one panel when motion and/or heat are detected.

17. The disinfecting light fixture of claim 16, wherein the sensors enable an opening of the at least one panel when motion and heat are no longer detected.

18. The disinfecting light fixture of claim 16, wherein the sensors deactivate the one or more UVC irradiation elements when motion and/or heat are detected.

19. The disinfecting light fixture of claim 18, wherein the sensors enable a reactivation of the one or more UVC irradiation elements when motion and heat are no longer detected.

20. The disinfecting light fixture of claim 16, wherein the disinfecting light fixture is mounted to a ceiling in the room and includes an adjustment mechanism for varying a distance between the disinfecting light fixture and the ceiling.
